(19) United States
(12) Patent Application Publication
(10) Pub. No.: US 2023/0003713 A1
(43) Pub. Date: Jan. 5, 2023

(54) METHODS OF SCREENING COMPOSITIONS FOR CANNABINOIDS

(71) Applicant: CCG Holding, Inc., San Diego, CA (US)

(72) Inventors: Dale HUNT, Escondido, CA (US); Rick BRENNER, Orlando, FL (US); Jian-Nong MA, Carlsbad, CA (US)

(21) Appl. No.: 17/756,745

(22) PCT Filed: Dec. 2, 2020
(86) PCT No.: PCT/US2020/062818

§ 371 Date: Jun. 1, 2022

Related U.S. Application Data
(60) Provisional application No. 62/942,602, filed on Dec. 2, 2019.

(51) Int. Cl. G01N 33/50; G01N 33/94; A01H 1/04; A01H 6/28

(52) U.S. Cl. CPC G01N 33/5008; G01N 33/948; A01H 1/04; A01H 6/28

(57) ABSTRACT

The invention generally relates to methods of screening a sample for cannabinoids.

### CLAIM OF PRIORITY UNDER 35 U.S.C. § 119

The present Application for Patent claims priority to Provisional Application No. 62/942,602 entitled “METHODS OF SCREENING COMPOSITIONS FOR CANNABINOIDS” filed Dec. 2, 2019, assigned to the assignee hereof and hereby expressly incorporated by reference herein.

### BACKGROUND

### Field

The invention generally relates to methods of screening a sample for cannabinoids.

### Background

The cannabinoids are a class of related C₂₁ terpenophenolic chemical compounds that act on cannabinoid receptors. Chemically, cannabinoids as a class have tremendous structural variability and are divided into ten subclasses on the basis of shared structural features. The identifying structure of cannabinoids is a prenylated polyketide backbone resulting from the combination of a fatty acid with an isoprenoid. In many cases, this initial reaction involves olivetolic acid and geranyl diphosphate to produce cannabigerolic acid (CBGA) that serves as a precursor for cannabinoid subclasses. The subclasses are differentiated by cyclization by a specific oxidocyclase that acts on CBGA to form the resulting cannabinoid acid. At least one additional mechanism has been identified for biosynthesis of phytocannabinoids in Cannabis. The propyl cannabinoids, such as tetrahydrocannabivarin (THCV), are identified by the presence of a C3 side chain instead of a C5 side chain and synthesized from a divarinic acid precursor. Unique varieties within the subclasses are identified by chemical group at certain side chains (Brenneisen, R., Marijuana and the Cannabinoids Forensic Science and Medicine. 2007). While mammals naturally produce endocannabinoids, phytocannabinoids produced by plants of the genus Cannabis have been used for their medicinal and psychoactive properties for millennia. In concert with other Cannabis biomolecules, cannabinoids interact with receptors in the human body to produce an “entourage effect” with an immense variety of possible physiological outcomes (Russo, 2019). As an alternative to the whole-plant entourage effect, there has been much energy and effort placed upon isolating individual cannabinoids.

The endocannabinoid system (ECS) has received renewed interest as novel roles for individual cannabinoids in appetite, neurology, pain, inflammation, and other responses have been identified. Interest in the ECS has driven further investigation for novel cannabinoids isolated from Cannabis. There are differences in the effects of these cannabinoids, and most of these differences are not yet fully characterized, indicating a lack of understanding of their mechanisms of action and the pathways that regulate the ECS. The classical cannabinoid receptors are the G-protein couple receptors (GPCRs), CB1 and CB2, and have been studied in the greatest detail. Additional receptors having an affinity for cannabinoids have been identified including orphan GPCRs (GPR3, GPR6, GPR12 (Laun, 2019); GPR18, GPR55, GPR119 (Ramirez-Oroxco, 2019)) and the transient receptor potential (TRP) channel TRPV1 (Muller, et al. Frontiers in Molecular Neuroscience 2019).

The most notable phytocannabinoids are tetrahydrocannabinol (THC), the primary psychoactive compound in Cannabis, and cannabidiol (CBD). Each of these cannabinoids has already become an industry unto itself, as reflected by the variety of products incorporating them, the number of companies formed to commercialize these products, the investments in such products and companies have attracted, and the revenues they have yielded in competitive markets. A few other “minor cannabinoids” have been isolated and are being characterized in terms of their physiological effects, and each of them also promises to spawn further massive industrial activity and medical insights. The extensive cultivation of Cannabis has led to the development of numerous genetic variants (genovars) within the species. These genovars have been selected over time for specific phenotypic traits including cannabinoid production. The heredity of cannabinoids is attributable to the inheritance of specific cannabinoid acid synthases that catalyze the enzymatic processes for cannabinoid production. The primary known cannabinoid synthases are THCA synthase (THCAS) and CBDA synthase (CBDAS) producing the cannabinoid acid forms of THC and CBD, respectively. Genetic studies have suggested that additional genes may regulate the total cannabinoid production and specific chemovars produced (Weiblen, 2015). To date, there are at least 113 different known cannabinoids. However, only a small fraction of these have been isolated in sufficient amounts and with sufficient purity to permit their characterization in terms of their receptor activation and/or their physiological effects.

### SUMMARY

Methods of screening samples for cannabinoids are provided. Such methods can be useful for groups such as cannabis breeders and medical researchers.

Some embodiments of the invention relate to a method of screening a composition for the presence of a cannabinoid. The method can include providing cells expressing at least one cannabinoid-activated receptor, adding a composition to the cells, and measuring a characteristic of the cells in the presence of the composition. In some embodiments, the characteristic can result from an interaction between the composition and at least one of the receptors and can be indicative of the presence of a cannabinoid. In some embodiments, the method can include identifying the presence of a cannabinoid in the composition based on the results of the measuring step.

Some embodiments of the invention relate to a high-throughput method of screening a composition for the presence of a cannabinoid. The method can include providing a first group of cells expressing a first cannabinoid-activated receptor and a second group of cells expressing a second cannabinoid-activated receptor, adding the composition to the first group of cells and second group of cells, and measuring a characteristic of the cells in the presence of the composition. In some embodiments, the characteristic can result from an interaction between the composition and at least one of the said receptors and can be indicative of the presence of a cannabinoid. In some embodiments, the method can include identifying the presence of a cannabinoid in the composition based on the results of the measuring step. In some embodiments there can be 3, 4, 5, 6, 7, or more groups of cells each expressing a different cannabinoid-activated receptor.

Some embodiments of the invention relate to a high-throughput method of screening multiple compositions for the presence of a cannabinoid. The method can include providing a first group of cells expressing a first cannabinoid-activated receptor, a second group of cells expressing a second cannabinoid-activated receptor; adding each of the multiple compositions to the first group of cells and second group of cells such that each group of cells is in contact with one of the multiple compositions and measuring a characteristic of the cells in the presence of the each of the multiple compositions. In some embodiments, the characteristic can result from an interaction between each of the multiple compositions and at least one of the receptors and can be indicative of the presence of a cannabinoid. In some embodiments, the method can include identifying the presence of a cannabinoid in one or more of the multiple compositions.

In some embodiments, a single group of cells can co-express multiple cannabinoid-activated receptors, with the other steps of the method being applied to the single group of cells either with or without other steps involving other groups of cells.

In some embodiments, the method of screening a composition for cannabinoid-like activity can include plotting relative signaling profiles to establish correlation.

In some embodiments, the compositions in the methods described can be a plant extract. If multiple compositions are used in a method, the multiple compositions can be plant extracts, and each of the multiple compositions can be a plant extract from a different part of the same plant. The plant extract can be an extract from any plant tissue including, but not limited to, flower trichomes, root, young plant, seedling, trichomes from other parts of plant that are not flower, and/or the like.

In some embodiments, the cannabinoid in the methods described can be a minor or an unknown cannabinoid. A cannabinoid not previously isolated, purified, and studied for its effects can be referred to as a “new” cannabinoid even if such cannabinoid had been previously identified, named and structurally characterized. The properties that define whether a cannabinoid is new or unknown are the properties of availability in some sort of commercial scale and understanding of the physiological, neurological, psychoactive, and/or pharmacological effects of the new cannabinoid, alone or in combination with other cannabinoids and/or other components of a plant such as, but not limited to, terpenes and flavonoids.

In some embodiments, the cannabinoid-activated receptor in the methods described above can be CB1, CB2, GPR18, GPR55, GPR119, TRPV1 or any other receptor activated known to be activated by a cannabinoid. Receptors can also include non-CB1/CB2 receptors or orphan GPCRs with limited sequence homology to CB1/CB2 such as GPR3, GPR6, GPR12, GPR18, GPR35, and GPR55. Receptors can also include some well-established GPCRs like the serotonin receptor 5-HT1A, alph2-adrenoceptor, opioid receptors, transient receptor potential channels (TRPV1-4, TRPM8, TRPA1), and PPARg for testing possible cross-talks with CB1/CB2 receptors and off-target activity.

In some embodiments, the measured characteristic in the methods described above can be a binding affinity of the composition to the receptor, a change in intracellular levels indicative of activation of the cannabinoid-activated receptor, a change in a secondary messenger indicative of activation of the cannabinoid receptor, phosphorylation of a molecule indicated of activation of the cannabinoid-activated receptor and/or the like. The characteristic is any cellular, physiological, genetic, regulatory, or chemical characteristic that is changed by the presence of a cannabinoid.

In some embodiments, the characteristic is the intracellular concentration of cAMP, Ca2+, or any other molecule whose concentration is changed by the presence of a cannabinoid.

In some embodiments, the characteristic is phosphorylation of a molecule that is changed by the presence of a cannabinoid. For example, the characteristic can be a change in the phosphorylation is of 38-MAPK, ERK, and/or any other molecule whose phosphorylation is affected by the presence of a cannabinoid.

In some embodiments, the characteristic is beta-arrestin recruitment. This can be measured by BRET (bioluminescence resonance energy transfer) assays or using commercially available in vitro technologies such as PathHunter beta-Arrestin Assay (DiscoverX), Tango GPCR Assay System (Thermo Fisher Scientific), and LinkLight/beta-arrestin Signaling Pathway Assay (BioInvenu).

In some embodiments, the method can employ a reporter gene assay system. A reporter gene can be synthesized in response to activation of a specific signaling cascade, followed by monitoring the activity of reporter gene expression. A luciferase reporter gene assay platform can be used as high throughput homogenous assay for screening GPCR targets due to its high sensitivity and reliability. The CB1/CB2 and related GPCRs and other orphan receptors couple to multiple G proteins that regulate respective downstream signaling pathways which eventually induce reporter gene transcription by various response elements such as CRE, NFAT, SRE, etc.

In some embodiments, the extracting step can include purifying one or more cannabinoid-containing fractions obtained from plant tissue employing separations techniques known in the art such as, for example, chromatography, isoelectric separations, dialysis, filtration, ultrafiltration, salting-out, differential centrifugation, and the like; testing aliquots from fractionated samples to determine which fraction contains a cannabinoid; further isolation of cannabinoid and confirmation of signaling profile after extraction; and/or using an extracted portion verified to be containing cannabinoid for further fractionation and/or purification and/or characterization studies of properties of the cannabinoid.

In some embodiments, the methods described above can include isolating the cannabinoid in purified or semi-purified form. After isolation, the method can include testing the isolated cannabinoid for activity. The testing can include receptor-binding assays, biochemical studies, and cellular and/or organism-level studies in any of a number of model systems including mammalian systems, or any other assay that can elucidate and/or confirm activity.

For example, the testing can include propagating a plant as a clone; preparing extracts in larger scale, wherein the extracts correspond to the fraction(s) originally found to contain receptor-activation activity; providing cells expressing at least one cannabinoid-activated receptor; adding the composition to the cells to verify that the signaling profile matches or corresponds to the initial result. The testing can also include performing DNA sequencing on the clone to identify a gene for unknown/minor/new cannabinoid.

For example, the testing can include administering the cannabinoid to mammalian cells; measuring characteristics of cells within organoid culture system relating to cannabinoid signal transduction; isolating total RNA from organoid culture to identify upregulated genes in response to cannabinoid administration. In some embodiments, the testing can include administering the novel cannabinoid to an animal model; measuring characteristics of cells within animal neural or other target tissue relating to cannabinoid signal transduction; isolating total RNA from animal tissues to identify upregulated genes in response to cannabinoid administration.

The testing can also include combining the cannabinoid with additional ingredients to screen for the potential of the cannabinoid and the one or more additional ingredients to work synergistically or as allosteric modulators. In such tests, one or more additional ingredients can be added to the composition and tested as described above.

In some embodiments, the method described above can include further testing and/or characterization of the cannabinoid. The further characterization can include mass spectrometry or any other method to characterize structure. The further testing can include identifying potential therapeutic application(s).

### DETAILED DESCRIPTION

The present invention relates to methods to identify and characterize novel cannabinoids found within various genovars of Cannabis. The methods can include the establishment of high-throughput biochemical and molecular biology protocols for measuring the interaction with known cannabinoid receptors to generate a signal profile for individual cannabinoids, generation of a classification system for cannabinoids, identification of cannabinoids present in whole plants and specific tissues of plants, breeding and/or propagation of plants containing novel/minor cannabinoids for further characterization, and assays to establish the impact and physiological functions of cannabinoids interacting with mammalian cells expressing the target(s) of interest.

The cannabinoid in the methods described can be a minor or an unknown cannabinoid. A cannabinoid not previously isolated, purified, and studied for its effects can be referred to as a “novel” or “new” cannabinoid even if such cannabinoid had been previously identified, named and structurally characterized but had no any published biological effects at known cannabinoid-activated receptors. The properties that define whether a cannabinoid is new or unknown are the properties of availability in some sort of commercial scale and understanding of the physiological, neurological, psychoactive, and/or pharmacological effects of the new cannabinoid, alone or in combination with other cannabinoids and/or other components of a plant such as, but not limited to, terpenes and flavonoids.

The invention detailed herein relates to methodology to describe the individual interactions for cannabinoids and establish a system (an integrated database platform) for identification and classification of novel or minor cannabinoids extracted from plant material.

Methods of screening a composition for the presence of a cannabinoid are provided. The method can include contacting the composition or a fraction thereof to cells expressing at least one cannabinoid-activatable receptor or putative cannabinoid receptor or a related receptor, and measuring a characteristic of the cells in the presence of the composition, where the characteristic results from an interaction between the composition and at least one of the receptors and is indicative of the presence of a cannabinoid. The characteristics of the cells that are to be measured can be defined as cellular processes that are activated in response to the composition interacting with a cannabinoid receptor in an allosteric, inverse, agonistic or antagonistic manner. These can be intracellular processes. These include but are not limited to phosphorylation of intracellular secondary messenger proteins, release of intracellular calcium (Ca²⁺) stores, or release or synthesis of intracellular secondary messenger proteins.

Using the method described above, some embodiments of the invention relate to a high-throughput method of screening a composition for the presence of a cannabinoid using multiple compositions with multiple groups of cells. For example, the method can include using 1, 2, 3, 4, or more groups of cells each expressing a different cannabinoid-activatable receptor. One or more multiple compositions can be tested at once with all the groups of cells. In other embodiments, the method can involve one or more single cell lines co-expressing multiple relevant receptors and can, for example, be run in multiplexed format.

Some embodiments of the invention relate to a method of isolating a cannabinoid from a plant or screening a plant for the presence of a cannabinoid. The method can include extracting a composition from the plant or from a selected plant tissue, optionally fractionating the extract thus produced to form one or more composition fractions, and contacting cells expressing at least one cannabinoid-activated receptor with the composition and/or fraction(s), and measuring a characteristic of the cells in the presence of the composition, where the characteristic results from or is indicative of an interaction between the composition and at least one of the receptors and is indicative of the presence of a cannabinoid. If the presence of a cannabinoid is indicated, then it can be determined that the extract or fraction contained a cannabinoid and further analysis and use of conventional biochemistry techniques can lead to isolation of the cannabinoid. The isolated cannabinoid can be further concentrated, purified, characterized and tested. Depending on the characterization and testing of the cannabinoid, the plant from which it was extracted may be selected for breeding purposes (i.e., to breed a plant that produces a novel or minor cannabinoid or any cannabinoid with a desired activity profile).

Further testing can include testing the cannabinoid(s) for potential therapeutic application. For example, the cannabinoid(s) can be tested for anti-cancer activity on a panel of human cancer cell lines (endogenously expressing cannabinoid related receptors) by measuring cell viability and cytotoxicity or anti-inflammatory and neuroprotective effects on microglia and SH-SY5Y neuroblastoma cell lines.

Some embodiments of the invention relate to a method of breeding a new plant variety based on the finding of (a) cannabinoid(s) in the methods described above. The method can include extracting a composition or compositions from a potential plant suitable for breeding and analyzing the composition or compositions using the methods described above. The method can also include breeding to obtain additional genetic combinations related to the cultivar in which the new cannabinoid was found, and screening multiple individuals from the breeding to identify one or more individuals expressing a higher amount of the new cannabinoid. Such breeding, screening, and selection can be continued according to conventional plant-breeding protocols and/or guided by non-traditional methods as well as molecular-biology methods, with the goal being the development of one or more cultivars expressing quantities of the new cannabinoid that are sufficient to enable practical and cost-effective extraction and study of the new cannabinoid. In some embodiments, breeding can include the use of genetic markers associated with desirable traits. Such marker-assisted breeding is known in the art, and selection of appropriate markers based upon desired traits is within the level of those of skill in the art.

In other embodiments, enhancement of expression and/or accumulation of a given minor or rare cannabinoid can require or be aided by molecular approaches. These include but are not limited to identification of a gene associated with expression level of a given cannabinoid and manipulating the expression of the gene by such approaches as increasing copy number, promoter modification, suppressor removal, elevation of expression of genes involved in accumulation of biosynthetic precursor molecules, depressing the expression of genes associated with biosynthesis of other cannabinoids that would otherwise act as a drain on precursor molecules, and the like. In still other embodiments, enhancement and/or accumulation of a given minor or rare cannabinoid can involve use of gene-editing tools such as, for example, CRISPR-Cas9 constructs, to directly elevate gene expression or otherwise facilitate biosynthesis and accumulation of the desired cannabinoid or group of cannabinoids.

Some embodiments of the invention relate to a method of classifying a cannabinoid or creating a classification system using the methods described above to create a signaling profile unique to the cannabinoid. The signaling profile can be used to create a reference library by assigning a reference value (e.g. the potency and/or efficacy) based on a known cannabinoid to the signaling profile and adding the reference value to a reference library. The reference library can be organized based on each signaling profile reference value to create a database of profiles of known cannabinoids.

With such an established library, a composition can be screened for cannabinoid-like activity by, for example, plotting relative signaling profiles to establish correlation. Correlation can be for pathway-specific or biased activation, etc. This database can be used to further facilitate identification and characterization of a heretofore unknown or uncharacterized cannabinoid. In some embodiments, this classification and organization of information can be enhanced with machine learning/artificial intelligence to generate predictive results as to the function, receptor targets, or other properties of a new/rare/unknown cannabinoid, as well as interactions between or among cannabinoids.

The methods disclosed herein can be oriented towards the discovery and molecular characterization of minor, unknown, or new cannabinoids and/or receptors. The methods disclosed herein can also be oriented towards selecting plants for breeding based on cannabinoid levels or cannabinoid activity. The methods disclosed herein further can be oriented towards establishing a way to classify cannabinoids, such as a library. The methods and/or the library can be useful for identifying and characterizing new or minor cannabinoids and/or identifying plants that produce new or minor cannabinoids and/or selecting plants for breeding based on cannabinoid levels, cannabinoid activity, cannabinoid activity profiles, and/or the like.

The methods and/or library can also be useful for identifying potential therapeutic applications of the new or minor cannabinoids. Examples of potential applications include, but are not limited to control of appetite, treatment/prevention of cancer, treatment/prevention of pain, treatment/prevention of neurodegenerative diseases, and/or the like.

### Compositions

The composition disclosed in this application can be any composition that can include a cannabinoid. The composition can be a plant extract. The plant extract can be an extract from a plant tissue such as whole flower, flower trichomes, root, young plant or portions thereof, leaves generally or certain types of leaves, meristems, seedling, trichomes from other parts of plant that are not flower, any tissue of the plant at any developmental stage, and/or the like.

The composition can also include the addition of one or more additional ingredients that may act as an allosteric modulator(s) or otherwise act synergistically with a cannabinoid in the composition. The one or more additional ingredients can include but are not limited to terpenes and flavonoids. Likewise, the receptor-based approach to detecting new ligands and characterizing the molecular and/or physiological effects of a given plant or extract from a plant can be extended to receptors for which terpenes, flavonoids, and other receptor ligands produced by the plant. As such, the techniques and approaches described herein can lead to numerous advances in understanding and characterizing the so-called “entourage effect” at molecular, cellular, and organismal levels. Any or all of these approaches can be combined with tools and techniques such as the use of organoids and/or machine learning to enhance the effectiveness and rate of progress toward the goal of fully understanding the function and potential of any given cannabinoid as well as the interactions of cannabinoids and other classes of plant molecules at the receptor level as well as in terms of gene regulation and downstream effects on cells and on organism physiology.

### Receptors

The receptors used in the methods disclosed herein can be any receptor that is activated by one or more cannabinoid, directly or indirectly. For example, the receptor can be CB1, CB2, GPR18, GPR55, GPR119, TRPV1, and/or any other cannabinoid-activatable receptor. For example, the receptor can be constitutively active orphan GRP3, GPR6 and GPR12 or some but not limited to functional heteromers of CB1/CB2, CB2/GPR18, and CB2/GPR55 (Morales, 2017). Some of the receptors can be tested simultaneously in multiplexed format and some form functional heteromers that can be tested as unique receptor complexes in the same way disclosed in the methods.

### Receptor Activity

Receptor activity can be based on any response in the cells that indicates receptor activation. For example, CB1, CB2 are GPCRs and their signaling pathways are known. Measurements of any of the known changes can be taken. Examples include changes in intracellular levels of various messengers or other components associated with messaging and/or receptor activation, changes in a secondary messenger activity, phosphorylation of a molecule, and/or the like. For example, intracellular levels of cAMP and/or Ca²⁺ can be measured. For example, p38-MAPK phosphorylation and pERK can also be measured. Likewise, reporter gene activation and related techniques can be employed to detect receptor activation and downstream signal transduction effects.

### Binding Affinity

Binding affinity of the cannabinoid or suspected cannabinoid can also be analyzed using methods and tools known in the art.

### Extraction

Compositions can be extracted from plants using methods known in the art. Extraction protocols can be selected that are suitable to the tissue from which the compositions are to be extracted. Extraction outcomes and extracts analyzed can range from crude extracts to extracts having passed through any of various separation and purification steps. In Cannabis, since cannabinoids are biosynthesized in the acid forms (THCA, CBDA etc.) which are usually less potent or inactive, suitable extraction protocols and efficient decarboxylation methods can be employed for optimal receptor responses (Lewis-Bakker, 2019). These conditions can differ for each Cannabis cultivar.

### Purification

Once a composition is identified as containing a cannabinoid, the cannabinoid can be further concentrated and purified by methods known in the art. Such methods can be adapted to the chemistry of a given cannabinoid. Separations and subfractionation techniques can employ any combination of chromatography, differential centrifugation, solvent/aqueous phase extraction, filtration, affinity extraction, and the like. One option is to separate an extract at any stage into aliquots containing molecular sub-populations having different properties, whether the differences be molecular weight, polarity, affinity, or the like. Testing aliquots from a fractionated extract can indicate which fraction contains a cannabinoid. After the cannabinoid is identified in a given aliquot, the properties of that aliquot can suggest further steps for larger-scale concentration or purification of the cannabinoid. In addition, further testing of receptor activation, chemical composition, chemical structure, and the like, can be done to confirm isolation and some of the properties of the isolated cannabinoid.

### Characterization

The structure of the cannabinoid can be determined by methods know in the art such as mass spectrometry, including MS techniques such as MALDI-TOF. The expression of cannabinoid synthases can also be assessed using DNA sequencing to compare gene expression at loci associated with production of specific cannabinoids. Cannabinoid synthases carry out the enzymatic processes to produce cannabinoids. The primary known cannabinoid synthases are THCA synthase (THCAS) and CBDA synthase (CBDAS) producing the cannabinoid acid forms of THC and CBD, respectively. Research has shown that genes associated with the cannabinoid synthesis pathway are distributed across the Cannabis genome. THCAS and CBDAS are differentially expressed in genovars with cannabinoid chemotype differences and there is evidence of gene duplication for cannabinoid synthase in the Cannabis lineage. The established genetic mapping of THCAS and CBDAS will allow for investigation of genetic variations at these loci associated production of novel or minor cannabinoids using similar sequencing methods. This sequence analysis allows identification of potential novel cannabinoid synthases with the identification of novel or minor cannabinoids. Other cannabinoid synthases and enzymes acting at other steps in cannabinoid biosynthesis are also known and are also available for use in these embodiments of the invention.

### Mammalian Studies

Known or new cannabinoids can be tested in mammalian cells, animal models, and volunteer human subjects to determine potential therapeutic and/or clinical uses. Notwithstanding the fact that no person has ever been documented to suffer a lethal overdose of Cannabis, appropriate safety and dosing protocols can be established at the outset of such testing in volunteer human subjects.

### EXAMPLE

### Example 1—High-Throughput Screening of Cannabinoids and Establishment of Database of Cannabinoid-Response Profiles

### A) Establishment of Cell Line Stably Expressing Cannabinoid Receptor(s).

HEK293 cells are cultured at 37° C. 5% CO₂ in Dulbecco's Modified Eagle Medium (DMEM) containing 10% Fetal Bovine Serum (FBS), 1% Penicillin-Streptomycin (Standard Growth Medium) for 7 days with regular passaging at 70% confluency. On day 7, cells are transfected with cannabinoid receptor-expressing plasmids or nucleic acids in other forms, at a pre-determined multiplicity of infection. Medium on cells is replaced with DMEM containing 10% FBS 8 ug/mL polybrene and a lentiviral construct expressing the desired cannabinoid receptor (see Table 1) and incubated for 18 hours at 37° C. 5% CO₂.

TABLE 1
Exemplary (non-limiting) list of cannabinoid
receptors to be transfected into cell lines for
biochemical assays of receptor signaling. The list
of receptors can also include additional cannabinoid
receptors and related receptors as needed.
Known and Putative Cannabinoid Receptors

CB1
CB2
GPR18
GPR55
GPR119
TRPV1

Cells are cultured for 3 days in standard growth medium at 37° C. 5% CO₂ following transfection and then plated to obtain single colonies by growing in selection medium and limiting dilutions. Colonies are screened for expression of the desired cannabinoid receptor(s) and positive clones identified by expression of GFP or similar marker contained within the plasmid lentiviral vector. Positive expression clones are expanded to create a stock which can be stored in liquid nitrogen for use in future experiments.

### B) Detection of Calcium (Ca²⁺) Flux Triggered by Cannabinoid Binding Cannabinoid Receptor(s).

Cells stably transfected with specific cannabinoid receptor(s) are cultured in standard growth medium at 37° C. 5% CO₂ in 96 well plates overnight. Ca²⁺ indicator is applied to cells for 1 hour at 37° C. and 5% CO₂ before addition of cannabinoid. One or more cannabinoids of interest are added to the cells and measurement of Ca²⁺ signal is immediately obtained using FlexStation™ or similar reading device (BD Biosciences). Readings are gathered over 2 minutes to generate a temporal response curve to the applied cannabinoid. For each cannabinoid receptor or combination of cannabinoid receptors, the signal intensity is recorded for each individual cannabinoid or mixture of cannabinoids. Untransfected HEK293 cells are used for negative controls to establish baseline. Incubations and detection are performed according to manufacturer's instruction.

C) Detection of Cyclic AMP (cAMP) Production Triggered by Cannabinoids Binding to Cannabinoid Receptor(s)

Procedures are done according to manufacturer specifications (PerkinElmer). In brief, cells stably transfected with specific cannabinoid receptor(s) are cultured in standard growth medium at 37° C. 5% CO₂ in 96 well plates overnight. Cannabinoids of interest are added to the cells for 30 min followed by addition of the cAMP detector and measurement using fluorescence resonance energy transfer. For each cannabinoid receptor or combination of cannabinoid receptors, the signal intensity is recorded for each individual cannabinoid or mixture of cannabinoids. Untransfected HEK293 cells are used for negative controls to establish baseline. Forskolin, a known cAMP inducer, is used as a positive control for detection of cAMP.

D) Detection of Phosphorylated ERK (pERK) from Cannabinoid Receptor Signaling

Cells stably expressing specific cannabinoid receptor(s) are plated in 96 well plates for 24 hours in standard growth medium before media is removed and replaced with serum-free DMEM containing 1 mg/mL Bovine Serum Albumin (BSA) for reduction of pERK background signal. Following 18 hours incubation in BSA containing DMEM, cannabinoid(s) of interest resuspended in DMEM are added to the cells and incubated at 37° C. for 5 min. Following 37° C. incubation, cells are moved to 4° C. ice bath and supernatant removed. 300 μL of lysis buffer are added per well and plate is placed on a shaker for 10 min before detection using the manufacturer's standardized detection protocol. For each cannabinoid receptor or combination of cannabinoid receptors, the signal intensity is recorded for each individual cannabinoid or mixture of cannabinoids. Untransfected HEK293 cells are used for negative controls to establish baseline.

### E) Detection of MAPK Phosphorylation in Response to Cannabinoid Binding to Cannabinoid Receptor

Cells stably expressing specific cannabinoid receptor(s) are plated in 96 well plates for 24 hours in standard growth medium at 37° C. before medium is removed and replaced with serum-free DMEM. Following 18 hours incubation at 37° C., cannabinoid(s) of interest resuspended in DMEM with 0.1% BSA are added to the cells and incubated at 37° C. for 5 min. Following 37° C. incubation, cells are moved to 4° C. ice bath and supernatant removed. 30 μL of lysis buffer are added per well and plate is placed on a shaker for 10 min. 10 μL of supernatant containing the lysate is then collected and added to a 96 well plate.

Detection reagents are added to the lysate according to the manufacturer's specifications and signal is determined using a compatible plate reader. For each cannabinoid receptor or combination of cannabinoid receptors, the signal intensity is recorded for each individual cannabinoid or mixture of cannabinoids. Untransfected HEK293 cells are used for negative controls to establish baseline.

F) Determination of Cannabinoid Affinity with Cannabinoid Receptor

Determination of receptor:ligand affinity is done using Biacore equipment per the manufacturer's protocol. Purified cannabinoid receptors are immobilized on a sensor chip in a Biacore apparatus for measurement. Purified cannabinoids are suspended in 100% ethanol at a concentration of 50 μM and flowed across the sensor at 50 μL/min.

A unique profile for signal transduction elicited by an individual cannabinoid as determined by the above methods, in any combinations, can be determined based upon measurements of Ca²⁺, cAMP production, ERK phosphorylation, and p38-MAPK phosphorylation. The difference in signals can further depend on the specific cannabinoid receptor(s) expressed by the cells for the measurements. The total results from the above methods for each cannabinoid as tested on cell lines expressing one or more cannabinoid receptor(s) can be cataloged and assigned a reference value. Table 2 is a list of many cannabinoids; any combination thereof can be used to establish a reference library of signals. As many plant extracts are likely to contain more than one cannabinoid, combinations of purified cannabinoids can be included in the establishment of the reference library. The reference values can be used for classification studies of cannabinoids isolated from plant extracts as outlined below.

TABLE 2
Non-limiting list of selected cannabinoids

| | | |
|---|---|---|
| Cannabigerolic acid (CBGA) | Cannabigerolic acid | Cannabigerol (CBG) |
| | monomehtylether (CBGAM) | |
| Cannabigerol | Cannabigerovarinic acid | Cannabigerovarin (CBGV) |
| monomethylether (CBGM) | (CBGVA) | |
| Cannabichromenic acid | Cannabichromene (CBC) | Cannabichromevarinic acid |
| (CBCA) | | (CBCVA) |
| Cannabichromevarin (CBCV) | Cannabichromevarin (CBCV) | Cannabidiolic acid (CBDA) |
| Cannabidiol (CBD) | Cannabidiol monomethylether | Cannabidiol-C₄ (CBD-C₄) |
| | (CBDM) | |
| Cannabidivarinic acid (CBDVA) | Cannabidivarin (CBDV) | Cannabidiorcol (CBD-C₁) |
| Delta-9-tetrahydrocannabinolic | Delta-9-tetrahydrocannabinolic | Delta-9-tetrahydrocannabinol |
| acid A (THCA-A) | acid B (THCA-B) | (THC) |
| Delta-9-tetrahydrocannabinolic | Delta-9-tetrahydrocannabinol- | Delta-9-tetrahydrocannabivarinic |
| acid-C₄ (THCA-C₄) | C₄ (THC-C₄) | acid (THCVA) |
| Delta-9-tetrahydrocannabivarin | Delta-9-tetrahydrocannabiorcolic | Delta-9-tetrahydrocannabiorcol |
| (THCV) | acid (THCA-C₁) | (THC-C₁) |
| Delta-7-cis-iso- | Delta-8-tetrahydrocannabinolic | Delta-8-tetrahydrocannabinol |
| tetrahydrocannabivarin | acid (Δ⁸-THCA) | (Δ⁸-THC) |
| Cannabicyclolic acid (CBLA) | Cannabicyclol (CBL) | Cannabicyclovarin (CBLV) |
| Cannabielsoic acid A (CBEA-A) | Cannabielsoic acid B (CBEA-B) | Cannabielsoin (CBE) |
| Cannabinolic acid (CBNA) | Cannabinol (CBN) | Cannabinol methylether (CBNM) |
| Cannabinol-C₄ (CBN-C₄) | Cannabivarin (CBV) | Cannabinol-C₂ (CBN-C₂) |
| Cannabiorcol (CBN-C₁) | Cannabinodiol (CBND) | Cannabinodivarin (CBVD) |
| Cannabitriol (CBT) | 10-Ethoxy-9-hydroxy-delta- | 8,9-Dihydroxy-delta-6a- |
| | 6a-tetrahydrocannabinol | tetrahydrocannabinol |
| Cannabitriolvarin (CBTV) | Ethoxy-cannabitriolvarin (CBTVE) | Dehydrocannabifuran (DCBF) |
| Cannabifuran (CBF) | Cannabichromanon (CBCN) | Cannabicitran (CBT) |
| 10-Oxo-delta-6a-tetrahydrocannabinol | Delta-9-cis-tetrahydrocannabinol | 3,4,5,6-Tetrahydro-7-hydroxy- |
| (OTHC) | (cis-THC) | alpha-alpha-2-trimethyl-9-n- |
| | | propyl-2,6-methano-2H-1- |
| | | benzoxocin-5-methanol (OH-iso-HHCV) |
| Cannabiripsol (CBR) | Trihydroxy-delta-9- | |
| | tetrahydrocannabinol (triOH-THC) | |

### Example 2—Screening of Plants for Cannabinoid Production

### A) Generation of Plant Isolates for Cannabinoid Receptor Signaling Assays

A sample of plant material (flower, leaf, trichome, root, meristem, seedling) is ground in a sterile mortar and pestle. 200 mg of the ground plant tissue is combined with 25 mL of 80% methanol in H2O to create a suspension. The suspension is vortexed for 30 seconds followed by 15 minutes of sonication. The resulting solution is filtered through a 0.22 μm Teflon filter and used for analysis or stored at −80° C. for later analysis. The plant material isolated in this manner is hereafter referred to as plant tissue extract; this basic technique and/or variations upon it that are standard within the art, can be used to prepare extracts from any and all possible sources of plant tissue.

### B) Detection of Calcium (Ca²⁺) Flux Triggered by Cannabinoid Binding Cannabinoid Receptor(s).

Cells stably transfected with specific cannabinoid receptor(s) are cultured in standard growth medium at 37° C. 5% CO2 in 96 well plates overnight. Ca²⁺ indicator is applied to cells for 1 hour at 37° C. and 5% CO2 before addition of test material. Plant tissue extract is added to the cells and measurement of Ca²⁺ signal is immediately obtained using FlexStation™ or similar reading device (BD Biosciences). Readings are gathered over 2 minutes to generate a temporal response curve to the applied cannabinoid. For each cannabinoid receptor or combination of cannabinoid receptors, the signal intensity is recorded for each plant tissue extract. Untransfected HEK293 cells are used for negative controls to establish baseline. Incubations and detection are performed according to manufacturer's instruction.

C) Detection of Cyclic AMP (cAMP) Production Triggered by Cannabinoid Binding Cannabinoid Receptor(s)

Procedures are done according to manufacturer specifications (PerkinElmer). In brief, cells stably transfected with specific cannabinoid receptor(s) are cultured in standard growth medium at 37° C. 5% CO2 in 96 well plates overnight. Plant tissue extracts are added to the cells for 30 min followed by addition of the cAMP detector and measurement using fluorescence resonance energy transfer. For each cannabinoid receptor or combination of cannabinoid receptors, the signal intensity is recorded for each plant tissue extract. Untransfected HEK293 cells are used for negative controls to establish baseline. Forskolin, a known cAMP inducer, is used as a positive control for detection of cAMP.

D) Detection of Phosphorylated ERK (pERK) from Cannabinoid Receptor Signaling

Cells stably expressing specific cannabinoid receptor(s) are plated in 96 well plates for 24 hours in standard growth medium at 37° C. before medium is removed and replaced with serum-free DMEM containing 1 mg/mL Bovine Serum Albumin (BSA) for reduction of pERK background signal. Following 18 hours incubation in DMEM with BSA, plant tissue extracts to be tested diluted in DMEM are added to the cells and incubated at 37° C. for 5 min. Following 37 C incubation, cells are moved to 4° C. ice bath and supernatant removed. 30 μL of lysis buffer are added per well and plate is placed on a shaker for 10 min before detection using the manufacturer's standardized detection protocol. For each cannabinoid receptor or combination of cannabinoid receptors, the signal intensity is recorded for each individual plant tissue extract. Untransfected HEK293 cells are used for negative controls to establish baseline.

### E) Detection of MAPK Phosphorylation in Response to Cannabinoid Binding to Cannabinoid Receptor

Cells stably expressing specific cannabinoid receptor(s) are plated in 96 well plates for 24 hours in standard growth medium at 37° C. before medium is removed and replaced with serum-free DMEM. Following 18 hours incubation at 37° C., plant tissue extract diluted in DMEM 0.1% BSA are added to the cells and incubated at 37° C. for 5 min. Following 37 C incubation, cells are moved to 4° C. ice bath and supernatant removed. 30 μL of lysis buffer are added per well and plate is placed on a shaker for 10 min. 10 μL of supernatant containing the lysate is then collected and added to a 96 well plate. Detection reagents are added to the lysate according to the manufacturer's specifications and signal is determined using a compatible plate reader. For each cannabinoid receptor or combination of cannabinoid receptors, the signal intensity is recorded for each individual plant tissue extract. Untransfected HEK293 cells are used for negative controls to establish baseline

### F) Analysis of Cannabinoids Present in Individual Cannabis Plants

The results from the above biochemical assays are compiled in total for each plant tested. The results are then compared to the previously established results for individual cannabinoids to determine the cannabinoids present within the plant extract. In the event that cannabinoid signaling profile for a plant extract does not match an established control or cannabinoid profile, additional purification steps are taken to isolate and analyze the cannabinoids found within the plant. Plant tissue extracts are handled using separations and subfractionation techniques that can employ any combination of chromatography, differential centrifugation, solvent/aqueous phase extraction, filtration, affinity extraction, and the like. Each fraction can be subjected to the above biochemical testing to identify any fraction(s) containing the cannabinoid. The cannabinoid is extracted from the fraction using, for example, sequential testing of subfractions isolated via high performance liquid chromatography (HPLC), column chromatography, or the like.

### Example 3—Propagation of Plants Featuring Unique Novel or Minor Cannabinoids

Plants that feature unique or interesting results from the biochemical assays for cannabinoid receptor activation are selected for further cultivation. Individual clones propagated for additional studies are used to generate tissue samples for further biochemical and genetic analysis.

Plant tissues including, for example, flower, leaf, trichome, root, and meristem are processed from a single clone as described above to extract cannabinoids. Extracted cannabinoids are added to cells stably expressing cannabinoid receptors to complete the assays described above.

### Example 4—Gene Reporter Assay

A cAMP responsive element (CRE)-based luciferase reporter assay is developed and validated for CB1/CB2 receptors. This is expanded to include a series of homogenous reporter assays using reporter vectors with different response elements (CRE, NFAT, SRE, a mutant form of SRE, etc.) built in upstream of luciferase gene to measure four major GPCR signaling pathways: a change in cAMP level, calcium mobilization, ERK/MPAK activation and small G protein RhoA activation, respectively using the same reporter assay format.

After validating the assays, the luciferase reporter gene assay panel is used to screen compounds against selected targets in agonist, antagonist and allosteric modulator mode. Compounds are tested for ability to potentiate or down-regulate agonist responses using different approaches including EC50 shift analysis and residual agonist activity.

Positive allosteric modulator (PAM) responses are obtained by co-incubation of EC20 ref ligand. Negative allosteric modulators (NAM) are also be identified by the ability to reduce ref ligand efficacy. In the presence of EC20 ref ligand, the responses are sufficient to pick up potential NAM activity if robust assays are used for the screenings.

Primary screens are done with 2 concentrations for each test compound if more test compounds are in queue. Confirmatory dose-response curves are used to rank order potencies. EC50 shift analysis is achieved by performing agonist dose responses in the presence and absence of test compound at varying concentrations.

Confirmatory tests are performed for signaling-specific biased ligands. The lead compounds are assessed across the wide range of downstream signaling pathways, both G protein-dependent and independent, to determine the mechanism of action and potential pathway-specific biased ligands or functional selectivity.

BRET-based beta-arrestin recruitment assay and Cisbio's HTRF assays for phosphor-ERK (Gi/o mediated), -AkT (Gbetagamma mediated), -CREB (cAMP/PKA mediated), and -NFkB (inflammation related) are used to determine signaling specific activation.

Off-target activity is determined by running lead compounds in SafetyScreen44 Panel (Eurofins) for safety profiling and lead optimization.

The various methods and techniques described above provide a number of ways to carry out the application. Of course, it is to be understood that not necessarily all objectives or advantages described are achieved in accordance with any particular embodiment described herein. Thus, for example, those skilled in the art will recognize that the methods can be performed in a manner that achieves or optimizes one advantage or group of advantages as taught herein without necessarily achieving other objectives or advantages as taught or suggested herein. A variety of alternatives are mentioned herein. It is to be understood that some embodiments specifically include one, another, or several features, while others specifically exclude one, another, or several features, while still others mitigate a particular feature by including one, another, or several other features.

Furthermore, the skilled artisan will recognize the applicability of various features from different embodiments. Similarly, the various elements, features and steps discussed above, as well as other known equivalents for each such element, feature or step, can be employed in various combinations by one of ordinary skill in this art to perform methods in accordance with the principles described herein. Among the various elements, features, and steps some will be specifically included and others specifically excluded in diverse embodiments.

Although the application has been disclosed in the context of certain embodiments and examples, it will be understood by those skilled in the art that the embodiments of the application extend beyond the specifically disclosed embodiments to other alternative embodiments and/or uses and modifications and equivalents thereof.

In some embodiments, any numbers expressing quantities of ingredients, properties such as molecular weight, reaction conditions, and so forth, used to describe and claim certain embodiments of the disclosure are to be understood as being modified in some instances by the term “about.” Accordingly, in some embodiments, the numerical parameters set forth in the written description and any included claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the application are approximations, the numerical values set forth in the specific examples are usually reported as precisely as practicable.

In some embodiments, the terms “a” and “an” and “the” and similar references used in the context of describing a particular embodiment of the application (especially in the context of certain claims) are construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application.

Variations on preferred embodiments will become apparent to those of ordinary skill in the art upon reading the foregoing description. It is contemplated that skilled artisans can employ such variations as appropriate, and the application can be practiced otherwise than specifically described herein. Accordingly, many embodiments of this application include all modifications and equivalents of the subject matter recited in the claims appended hereto as permitted by applicable law. Moreover, any combination of the above-described elements in all possible variations thereof is encompassed by the application unless otherwise indicated herein or otherwise clearly contradicted by context.

All patents, patent applications, publications of patent applications, and other material, such as articles, books, specifications, publications, documents, things, and/or the like, referenced herein are hereby incorporated herein by this reference in their entirety for all purposes, excepting any prosecution file history associated with same, any of same that is inconsistent with or in conflict with the present document, or any of same that may have a limiting effect as to the broadest scope of the claims now or later associated with the present document. By way of example, should there be any inconsistency or conflict between the description, definition, and/or the use of a term associated with any of the incorporated material and that associated with the present document, the description, definition, and/or the use of the term in the present document shall prevail.

In closing, it is to be understood that the embodiments of the application disclosed herein are illustrative of the principles of the embodiments of the application. Other modifications that can be employed can be within the scope of the application. Thus, by way of example, but not of limitation, alternative configurations of the embodiments of the application can be utilized in accordance with the teachings herein. Accordingly, embodiments of the present application are not limited to that precisely as shown and described.

### REFERENCES

Mallipeddi S et al.: Functional selectivity at G-protein coupled receptors: Advancing cannabinoid receptors as drug targets. Biochem Pharmacol. 2017 Mar. 15; 128:1-11.
Ibsen M S et al.: Cannabinoid CB1 and CB2 Receptor Signaling and Bias. Cannabis Cannabinoid Res. 2017 Mar. 1; 2(1):48-60.
Southern C et al.: Screening β-arrestin recruitment for the identification of natural ligands for orphan G-protein-coupled receptors. J Biomol Screen. 2013 June; 18(5):599-609.
Wang T et al.: Measurement of β-Arrestin Recruitment for GPCR Targets. In: Sittampalam G S et al.: Assay Guidance Manual [Internet]. Bethesda (Md.): Eli Lilly & Company and the National Center for Advancing Translational Sciences; 2004-.
Cheng Z et al.: Luciferase Reporter Assay System for Deciphering GPCR Pathways. Curr Chem Genomics. 2010 Dec. 21; 4:84-91.
Liu B, Wu D.: Analysis of the coupling of G12/13 to G protein-coupled receptors using a luciferase reporter. Methods Mol Biol. 2004; 237:145-9.
Laun A S et al.: GPR3, GPR6, and GPR12 as novel molecular targets: their biological functions and interaction with cannabidiol. Acta Pharmacol Sin. 2019 March; 40(3):300-308.
Lewis-Bakker M M et al.: Extractions of Medical Cannabis Cultivars and the Role of Decarboxylation in Optimal Receptor Responses. Cannabis Cannabinoid Res. 2019 Sep. 23; 4(3):183-194.
Morales P, Reggio P H: An Update on Non-CB1, Non-CB2 Cannabinoid Related G-Protein-Coupled Receptors. Cannabis Cannabinoid Res. 2017 Oct. 1; 2(1):265-273.
Ramirez-Orozco R E et al.: Potential metabolic and behavioural roles of the putative endocannabinoid receptors GPR18, GPR55 and GPR119 in feeding. Curr Neuropharmacol. 2019; 17(10):947-960.
Russo E B: The Case for the Entourage Effect and Conventional Breeding of Clinical Cannabis: No “Strain,” No Gain. Front Plant Sci. 2019 Jan. 9; 9:1969.
Weiblen G D, Wenger J P, Craft K J, ElSohly M A, Mehmedic Z, Treiber E L, Marks M D. Gene duplication and divergence affecting drug content in Cannabis sativa. New Phytol. 2015 December; 208(4):1241-50.

1. A method of screening a composition for the presence of a cannabinoid, comprising:
providing cells expressing at least one cannabinoid-activated receptor;
adding said composition to the cells;
measuring a characteristic of the cells in the presence of the composition, wherein the characteristic results from an interaction between the composition and at least one of the said receptors and is indicative of the presence of a cannabinoid; and
identifying the presence of a cannabinoid in the composition based on the results of the measuring step.

2.-7. (canceled)

8. A method of breeding a plant variety comprising a cannabinoid, comprising:
extracting a composition from a potential plant suitable for breeding;
providing cells expressing at least one cannabinoid-activated receptor;
adding the composition to the cells;
measuring a characteristic of the cells in the presence of the composition, wherein the characteristic results from an interaction between the composition and at least one of the said receptors and is indicative of the presence of a cannabinoid;
identifying a plant suitable for breeding based on the results of the measuring step;
using the plant suitable for breeding to breed a plant variety comprising a cannabinoid.

9. A method of classifying a cannabinoid comprising
providing cells expressing at least one cannabinoid-activated receptor;
adding said cannabinoid to the cells;
measuring multiple characteristics of the cells in the presence of the cannabinoid, wherein each of the multiple characteristics results from an interaction between the cannabinoid and at least one of the said receptors;
using the characteristics based on the results of the measuring step to create a signaling profile; and
classifying the cannabinoid based on the signaling profile.

10. (canceled)

11. (canceled)

12. The method of claim 1, wherein the composition is a plant extract.

13. (canceled)

14. (canceled)

15. The method of claim 1, wherein the cannabinoid is a minor or an unknown cannabinoid.

16. The method of claim 1, wherein the at least one cannabinoid-activated receptor is selected from the group consisting of CB1, CB2, GPR18, GPR55, GPR119, and TRPV1.

17. The method of claim 1, wherein the characteristic is selected from the group consisting of: a binding affinity of the composition to the receptor, a change in intracellular levels indicative of activation of the cannabinoid-activated receptor, and a change in a secondary messenger indicative of activation of the cannabinoid receptor, phosphorylation of a molecule indicated of activation of the cannabinoid-activated receptor.

18. The method of claim 17, wherein the concentrations are one or more of cAMP and/or Ca²⁺.

19. The method of claim 17, wherein the phosphorylation is of p38-MAPK phosphorylation, pERK.

20. (canceled)

21. The method of claim 1, further comprising isolating the cannabinoid

22.-27. (canceled)

28. The method of claim 1, wherein the cannabinoid is further characterized.

29. The method of claim 28 wherein the characterizing step comprises mass spectrometry.

30. The method of claim 8, wherein the cannabinoid is a minor or an unknown cannabinoid.

31. The method of claim 8, wherein the at least one cannabinoid-activated receptor is selected from the group consisting of CB1, CB2, GPR18, GPR55, GPR119, and TRPV1.

32. The method of claim 8, wherein the characteristic is selected from the group consisting of: a binding affinity of the composition to the receptor, a change in intracellular levels indicative of activation of the cannabinoid-activated receptor, and a change in a secondary messenger indicative of activation of the cannabinoid receptor, phosphorylation of a molecule indicated of activation of the cannabinoid-activated receptor.

33. The method of claim 32, wherein the concentrations are one or more of cAMP and/or Ca²⁺.

34. The method of claim 32, wherein the phosphorylation is of p38-MAPK phosphorylation, pERK.

35. The method of claim 9, wherein the cannabinoid is a minor or an unknown cannabinoid.

36. The method of claim 9, wherein the at least one cannabinoid-activated receptor is selected from the group consisting of CB1, CB2, GPR18, GPR55, GPR119, and TRPV1.

37. The method of claim 9, wherein the characteristic is selected from the group consisting of: a binding affinity of the composition to the receptor, a change in intracellular levels indicative of activation of the cannabinoid-activated receptor, and a change in a secondary messenger indicative of activation of the cannabinoid receptor, phosphorylation of a molecule indicated of activation of the cannabinoid-activated receptor.
